(19) United States
(12) Patent Application Publication
(10) Pub. No.: US 2010/0021567 A1
(43) Pub. Date: Jan. 28, 2010

(54) Peganine Isolated from Anisotes Trisulcus L. as a Smoking Deterrent and Anorxigenic Agent

(76) Inventors: Adnan Jathlan Al-Rehaily, Riyadh (SA); Mansour Sulaiman AL-Said, Riyadh (SA); Kamal Eldin Hussein EL Tahir, Omdorman (SD)

Correspondence Address: Al-Rehaily Adnan Jathlan;Associate Professor, Department Pharmacognosy, College of Pharmacy, King Saud University, P O Box 2457, Riyadh, 11451, SA

(21) Appl. No.: 11/992,608

(22) PCT Filed: Nov. 7, 2006
(86) PCT No.: PCT/IB2006/002059

§ 371 Date: Mar. 27, 2008

(51) Int. Cl. A61K 36/19; A61K 31/502; A61P 3/00; A61P 25/34

(52) U.S. Cl. 424/725; 514/248

(57) ABSTRACT

This patent concerns the discovery of the effectiveness of an alcoholic total extract of the aerial parts of the plant Anisotes trisulcus (Forssk.) Vahl family Acanthaceae and its active constituent peganine as a new means for suppression of tobacco intake and as a suppression to appetite (as anororexiants). The treatment of Wistar rats with the total alcoholic extract in doses of 600 mg/100 ml drinking water decreased oral nicotine intake by 87.7% and food intake by 84.9%. The active constituent peganine in doses of 1.4 mg/ml drinking water decreased nicotine consumption by 79% and food intake by 77.8%. In other experimental in mice, the extract enhanced nicotine actions by more than 70% and mimicked nicotine in inducing intestinal stimulation in the isolated guinea pig ileum. Peganine is hereby claimed to be a new deterrent for tobacco intake.

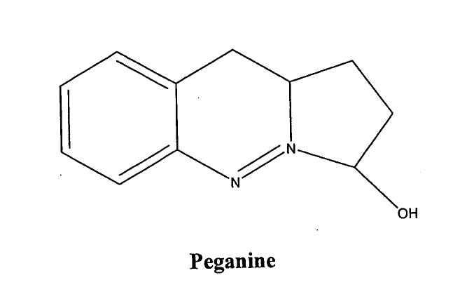

Throughout the world, various people almost 20% of the whole population enjoy tobacco actions via either smoking [cigarettes, pipes or shisha (nargilla)] or its sublingual intake under the tongue in a semi-dry form moistened with sodium bicarbonate (or as snuff) e.g. in Sudan and a some other African countries (1).

Chronic consumption of tobacco can produce two types of adverse reactions, namely cancer of lungs and throats when it is smoked due to the tar components and a group of diseases such as hypertension, arrhythmias, hyperlipidemias, asthma and gastric and duodenal ulcers together with development of dependence with the consequence maintenance of intake due to its nicotine content (1-3). These disorders usually predispose to death.

Nicotine usually mediates its actions via two types of main receptors located at two sites in the body namely postsynaptically in the neuromuscular junction or the skeletal muscles and in the neuronal tissues peripherally and centrally at the ganglia and neurons. It excites these receptors to release acetylcholine (4). The neuronal nicotinic receptors are ion-gated channels each of which is composed of 17 subunits. Ten of them are α type, 4 are β type together with γ, δ and ε (epsilon) (4). The most important receptor that is involved in nicotine dependence and the support of maintenance of nicotine intake in its various forms is the one containing the subunits α₄ β₂ (5). Activation of these receptors releases dopamine via release of acetylcholine in the nucleus accumbens and the frontal cortex (6). The release of dopamine plays an important part in nicotine-induced dependence, toxic actions and addiction (7).

Many investigators throughout the world are involved in researches aiming for discovering of drugs to help in cessation of tobacco intake in its various forms. This patent describes for the first time a chemical namely the alkaloid peganine isolated from the aerial parts of Anisotes trisulcus (Forssk.) Vahl family Acanthaceae collected from the Southern region of Saudi Arabia (8) that proved to suppress nicotine consumption and thereby opening the avenue to place itself as one of the limited armenatum available today to help in cessation of tobacco intake.

Peganine is the major quinazoline alkaloid isolated from Anisotes trisulcus (Forssk.) Vahl (represented 23.26% yield from AT-2 fraction). The alkaloid is also previously isolated from different plant species (9-13). Besides peganine, the plant produced other alkaloidal constituents such as anisotine, vasicinone (14), trisulcusine and methoxypeganine (15). Anisotes trisulcus (Forssk.) Vahl is a shrub locally known as Almodh (16) and is a traditional herbal medicine in the Arabian Peninsula and found its way in the folk medicine of Saudi Arabia as an antidiabetic. The plant is also used as treatment for all hepatic conditions including hepatitis, jaundice and other hepatic problems (17-19). The pharmacognostical studies on the leaf of the plant have also been investigated (20). The alkaloid peganine was reported to possess abortifacient and uterine stimulant activity (21-23). In addition, it was shown to produce slight but persistent bronchodilatation, slight hypotension and to inhibit peristalsis of isolated gut (24). Other studies showed bronchoconstriction and a negative inotropic effect of peganine with reduced coronary flow in the isolated heart (24).

### MATERIALS AND METHODS

### Plant Material

Anisotes trisulcus (Forssk.) Vahl was collected in March, 2005 from the way toward Fifa Mountains, Saudi Arabia.

### Extraction

The dried aerial parts (1 kg) of A. trisulcus were exhaustively extracted by cold percolation with 90% EtOH. The combined EtOH extract was concentrated in vacuo at 40° C. to produce a solid residue (75 g). The alcoholic extract was dissolved in 225 ml of 2% tartaric acid and extracted with CHCl₃ to remove acidic and neutral components (8 g) (Fraction AT-1). The aqueous acidic solution was then alkalinized with dilute NH₄OH to pH 9 and extracted with CH₂Cl₂ to produce 0.688 g (Fraction AT-2). The aqueous layer was made more alkaline with NH₄OH to pH 12 and extracted with EtOAc to get 0.64 (Fraction AT-3). The remaining aqueous phase was made acidic with tartaric acid to pH 4 and then lyophilized to yield 6 g (Fraction AT-4).

### Isolation and Identification of Peganine

The fraction AT-2 (0.688 g) was chromatographed via chromatotron 2 mm plate using EtOH absol.:CHCl₃:NH₄OH (5:95:4 drops) to afford 160 mg of peganine (23.26% yield) that was crystallized using mixture of CHCl₃ and MeOH. The alkaloid was identified as peganine by comparing its physical and spectral data with those reported previously in our laboratory and with the literature (14).

### Preparation of the Alcoholic Extract for Biological Studies:

The total extract was suspended in 0.25% aqueous sodium carboxymethylcellulose to give a concentration of 12.5% (w/v) initially and then diluted with the same vehicle as appropriate.

### Determination of LD₅₀ of the Extract:

Swiss albino mice (25 g body weight) and albino Wistar rats (200 g body weight) were divided each into 5 groups (N=10 animals per group). Animals in the different groups were injected with the extract intraperitoneally to provide doses of 0.4, 0.8, 1.6, 2.4 and 3.2 g/kg in the different groups. The animals were observed for any changes in behavior or death continuously for 2 hours following the injection. Any alive animals were observed for 72 hours thereafter. The percentage of death in each group was calculated. The LD₅₀ values were then determined following the methods of Litchfield and Wilcoxon, (25); Paget (26) and Ghosh (27).

### Induction of Nicotine Convulsions:

Albino mice were divided into different groups (N=5) and injected with different doses of nicotine dissolved in water. The animals were injected (i.p) with doses 0.8, 1.6, 3.2 and 6.4 mg/kg. The animals were continuously observed for 2 hours for appearance of any changes in behavior such as tremors, convulsions (clonic or tonic), abnormal movements changes in tail position and shapes, changes in locomotor activity and death.

To investigate the effect of the extract on nicotine-induced convulsions and death a sub-effective dose of the extract, (a dose of 1 g/kg) that did not produce any observable changes in behavior was initially administered to a group of 5 naive mice. Then after 5 minutes nicotine LD₂₅ was then administered and the animals were observed continuously for any changes in behavior, convulsions or death. The time of convulsions and death were recorded.

### Effect of the Extract on Isolated Guinea-Pig Ileum:

Albino guinea-pigs (450 g body weight) were killed using excessive ether anesthesia. The abdomen was opened and pieces of ileum (2 cm-long) were cut and suspended in an organ bath containing oxygenated Krebs' solution (pH 7.4) at 37° C. One end of the tissue was attached to the bottom of the organ bath and the other end was attached to an isometric transducer (Myopraph F60 with 0.5 and 5 g tension calibrations, supplied by Narco Biosystems, Houston, USA) and attached to a universal coupler No. 7189 fitted into a Narco physiograph. To examine the effect of the extract, the latter was added to the fluid bathing the tissue in different doses and allowed to contact the tissue for 45 seconds. The produced effect was recorded. To examine the effect of the blockers on the induced contraction, the following procedure was used. Hexamethonium (at a dose of 10-100 μg/ml) or atropine (at a dose of 0.1-20 μg/ml) was added to the bathing fluid and allowed to contact the tissue for 5 minutes and then the submaximal dose of the extract was added. The percentage inhibition induced by the blocker on the extract-induced contractions was then calculated.

### Effect of the Total Extract, its Fractions and Peganine on Nicotine and Food Consumption in Rats:

Male Wistar rats (200 g body weight) were divided at random into various groups (N=6 animals per group). Each animal was placed in a stainless steel cage and provided with a known weight of normal rat chow food pellets and drinking bottle containing a known volume of aqueous 0.025% sodium carboxymethylcellulose containing either nicotine acid tartarate to provide a final concentration of 40 μg/ml (in case of control groups) or the same concentration of nicotine plus the indicated concentrations of the whole extract, one of its fractions or peganine. The latter were added in form of their emulsions in 0.25% aqueous sodium carboxymethylcellulose in volumes that provide a final concentration of 0.025% of sodium carboxymethylcellulose in the drinking fluid. The animals were allowed both food and the drinking liquid ad libitum. The room temperature where the animals were placed was maintained at 23±2° C. The light cycle was kept at 12 h light and 12 h dark. The relative humidity was maintained at 75±5%. Both food and drinking liquid consumptions were measured every 2 days and at the end of the treatment which continued for complete seven consecutive days. The influence of each treatment on food consumption, the drinking fluid intake and hence nicotine consumption was calculated at the end of the treatment.

### Statistical Analysis

All results were reported as their means±s. e. mean with N=number of experiments. Statistical significance between control and treated groups were performed using student's t-test.

### Results

### Effects of the Total Alcoholic Extract:

A) Lethal Dose 50 (LD₅₀): in rats and mice was 1.8 g/kg (i.p).
B) Lethal Dose (LD₁₀₀) in rats and mice was more than 2.4 g/kg (i.p).

Symptoms Before Death:

1—Increase in locomotor activity

2—Abdominal respiration (shallow respiration)

3—Shaking of tail

4—Convulsions

Onset of death following administration was 4-5 minutes.
C) Effect of the extract on nicotine-induced convulsions and death in mice:
Table 1 shows the central actions of nicotine alone in the dose range 1.6 to 6.4 mg/kg (i.p) in mice and the effect of the total extract in a sub-effective dose of 1 g/kg (i.p) on nicotine-induced actions. As it can be seen in table 1, the extract at a dose of 1 g/kg (i.p) into mice potentiated the actions of nicotine by 300%.
D) Effect of the total extract on the isolated guinea-pig ileum:
   1—Exposure of the guinea-pig ileum to the total extract in doses of 15, 30 and 60 μg/ml bathing fluid induced dose-dependent contractions.
   2—The stimulant effect was not blocked by either hexamethonium (100 μg/ml) or atropine (10 μg/ml).
      Conclusion: The stimulant effect of the extract was not due to activation of ganglia or muscarinic M₃ receptors.

### Effect of the Total Alcoholic Extract on Nicotine and Food Consumption in Rats:

in control rats provided with drinking water containing nicotine in a concentration of (40 μg/ml), the daily consumption of the drinking vehicle per 200 g rat was 26.1±1.3 ml and the daily food consumption was 23.9±0.9 g. Treatment of the animals with the total alcoholic extract in a dose of 600 mg/100 ml drinking vehicle reduced the nicotine consumption and food intake by 87.7 and 84.9%, respectively. The cumulative effects are shown in table 2.

### Effect of the Different Fractions of the Total Alcoholic Extract on Nicotine and Food Consumption in Rats:

Treatment of rats with each of the different fractions of the total alcoholic extract mixed with nicotine in the drinking vehicle revealed that only fraction AT-2 possessed some ability to suppress both nicotine and food intake. Table 3 shows the cumulative results. When higher concentrations of the different fractions were used, here again fraction AT-2 proved to be the most effective and produced significant reductions in both actions (see table 4).

### Effect of Peganine on Nicotine and Food Consumption in Rats:

As peganine was found to be the major constituent from the fraction AT-2 in this study, it was tested in rats. Treatment of the animals with peganine 1.4 mg/ml in the drinking vehicle mixed with nicotine (40 μg/ml) produced significant reduction in both nicotine and food consumptions. It decreased the nicotine consumption and food intake by 79 and 77.8%, respectively. The details are shown in table 5.

### DISCUSSION

The results of this study revealed that the extract of Anisotes trisulcus (Forssk.) Vahl induced significant inhibition in nicotine consumption, reduced the lethal dose of nicotine by 75% and mimicked nicotine in inducing intestinal contractions (probably via activation of a subtype of nicotine receptors in the guinea-pig intestinal ganglia-albeit not blocked by the ganglionic blocker hexamethonium). Furthermore, it induced significant suppression of food intake. Its active constituent peganine (vasicine or linarine) which is known chemically as 1,2,3,9-tetrahydropyrolo[2,1-b]quinazolin-3-ol produced potent significant inhibitions of nicotine intake and food consumption. It exerted a potent anorexigenic action. In fact this constituent acted as a nicotine substitute.

Peganine actions are probably mediated via activation of α₄ β₂ nicotinic receptors as does nicotine leading to release of dopamine in the mesolimbic sites of the brain. Thus, it probably acted as a nicotine substitute leading to satisfaction of the rewarding system leading to an explanation of the reduced nicotine intake (28).

Generally, speaking an agonist at a certain receptor may either be a potent, low potent partial or potent partial agonist. Previous pharmacological studies revealed that potent nicotinic agonists or low potency partial agonists would not be expected to antagonize the effects of nicotine but agents that act as potent partial agonists can antagonize nicotine (29-30). For instance vareniciline, the potent partial nicotinic α₄ β₂ agonist, in a dose of 5.6 mg/kg significantly blocked nicotine (1 mg/kg)-induced increase in dopamine turnover in the mesolimbic system of rats (30). Thus, since the extract containing peganine did not antagonize nicotine-induced convulsions and death but instead mimicked nicotine and reduced its LD₅₀ by >50% and in addition it substituted for nicotine in the nicotine drinking experiments it is plausible to suggest that peganine acted as does nicotine at the α₄ β₂ nicotinic receptor. Indeed, the types of convulsions observed before death following peganine were reminiscent to those of nicotine.

Thus, it is highly likely that peganine can act to limit craving and withdrawal from nicotine in chronic tobacco consumers (smokers or snuff intakers). Thus, we claim that peganine has the potential of a novel treatment for tobacco dependence. In this aspect, it is hoped to be more fruitful than the partial nicotinic agonist (−)-cytisine (31-32) that has poor blood brain barrier penetrability (33). It is hoped to be a new addition to the armenatum of tobacco cessation campaigns that include the skin patch containing both nicotine and its antagonist mecamylamine (34). The soonly expected varenicline, the partial α₄ β₂ nicotinic agonist (30), the antidepressant bupropion (or amfebutamine) (35) and the nicotine conjugate vaccine (Nicvax®) (36).

Considering food intake and appetite, we can recall that the two most important brain neurotransmitters that suppress food intake are dopamine and serotonin (37). The significant inhibitions induced by both extract and its constituent peganine in food intake incline us to believe that the anorexigenic effects were mediated by dopamine release via initial activation α₄ β₂ nicotinic receptors. Indeed activation of these receptors releases dopamine (6, 7, 28).

In conclusion, this patent showed that both of the extract of Anisotes trisulcus (Forssk.) Vahl family Acanthaceae and its constituent peganine possessed properties that enabled them to suppress nicotine consumption and food intake. Thus, we claim:
1—The use of the extract of Anisotes trisulcus (Forssk.) Vahl, family Acanthaceae in any pharmaceutical form as a method to limit tobacco consumption either via smoking, sublingually or snuffing.
2—The use of peganine in any pharmaceutical form as a means to limit tobacco consumption in any form smoking, sublingually or snuffing.
3—The use of the extract of Anisotes trisulcus (Forssk.) Vahl, family Acanthaceae in any pharmaceutical form as a means to suppress food intake.
4—The use of peganine in any pharmaceutical form as an anorexigenic agent.

### ACKNOWLEDGEMENT

We would like to thank Professor Farouk S. El-Feraly for his kind suggestions and encouragements. We would also like to thank the College of Pharmacy, King Saud University for providing the facilities to carry out this work.

### REFERENCES

1—E L Tahir, Kamal E. H. (1985). Cigarettes, Snuff and Health (1984) Publisher: Sudan Council of Research, Khartoum, Sudan.
2—Taylor, A. L. and Bettcher, D. W. (2000). Bull. World Health Organization 78, 920-929.
3—Doll, R., Peto, R., Boreham, J. and Sutherland, I. (2004). Mortality in relation to smoking: 50 years observations on male British Doctors. Br. Med. J. 328, 1519-1527.
4—Sargent, P. B. (2000). The distribution of neuronal nicotinic ACh receptors (eds. Clementi, F., Fornassari, D. and Gotti, C.) Publisher: Springer-Verlag, Berlin, Heidelberg, Germany, Vol. 144, 163-192.
5—Cohen, C. B., Bergis, O. E., Galli, F., Lockhead, A., Jegham, S., Biton, B., Leonardon, J., Avent, P., Sagard, F., Besnard, F., Graham, D., Coste, A., Oblin, A., Curet, O., Voltz, C., Gardes, A., Caille, D., Perrault, G., George, P., Sourbrie, P. and Scatton, B. (2003). SSR 591813, a novel selective and partial α₄ β₂ nicotinic receptor agonist with potential to smoking cessation. J. Pharmacol. Expt. Ther. 306, 407-420.
6—Dani, J. A. and De Biasi, M. (2001). Cellular mechanism of nicotine addiction. Pharmacol. Biochem. Behav. 70, 439-446.
7—Di Chiara, G. (2000). Role of dopamine in the behavioral actions of nicotine related to addiction. Eur. J. Pharmacol. 393, 295-314.
8—Collenette, S. (1999). Wildflowers of Saudi Arabia. Publisher: National Commission for Wildlife Conservation and Development, Riyadh, Kingdom of Saudi Arabia, p. 2.
9—Groger, D. and Johne, S. (1965). Occurrence of peganine in Linaria species. Planta Med. 13, 182.
10—Huq, M. E., Ikram, M. and Warsi, S. A. (1967). Chemical composition of Adhatoda vasica Linn-II, Pak. J. Sci. Ind. Res. 10, 224-225.
11—Ghosal, S., Chauhan, R. and Mehta, R. (1975). Chemical constituents of Malvaceae. Part 1. alkaloids of Sida cordifolia. Phytochemistry, 14, 830-832.
12—Kurbanov, D. and Zharekeev, B. K. (1974). Alkaloids of Beibersteinia multifida and Peganum harmala from the Karakalpak assr. Chem. Comp. 10, 715.
13—Schipper, A. and Volk, O. H. ((1960). The alkaloids of Peganum harmala. Dtsch. Apoth. Ztg. 100, 255.
14—Al-Azizi, M. M. (1997). Qinazoline alkaloids from Anisotes trisulcus L. Egypt J. Biotechnol. 2, 1-6.
15—Al-Rehaily, A. J., El-Sayed, K. A., Al-Said, M. S. and Ahmed, B. (2002). Trisulcusine: A novel spiro quinazoline alkaloid from Anisotes trisulcus. Indian Journal of Chemistry, 41B, 2385-2389.
16—Al-Hubaishi, A. and Muller-Hohenstein, K. (1984). Introduction to Vegetation of Yemen, Ecological Basis, Floristic Composition, Human Influence. p. 134 & 189.
17—Fleurentin, J., Hoefler, C., Lexa, A., Mortier, F. and Pelt, J. M. (1986). Hepatoprotective properties of Crepis rueppellii and Anisotes trisulcus: two traditional medicinal plants of Yemen. J. Ethnopharmacol. 16, 105-111.
18—Lanhers, M. C., Bertrand, I., Fleurentin, J., Lehr, P. R. and Pelt, J. M. (1986). J. Drug Research, 36, 826-829.
19—Al-Harbi, M. M., Al-Said, M. S. and Al-Azizi, M. M. (1992). Preliminary phytochemical and pharmacological studies on Anisotes trisulcus. Int. J. Pharmacognosy, 30, 87-92.
20—Al-Rehaily, A. J. (2000). Pharmacognostic studies on the leaf of Anisotes trisulcus (Forssk.). Pak. J. Bio. Sci. 3, 1427-1430.
21—Gupta, O. P., Sharma, M. L., Ray Ghaatak, B. J. and Atal, C. K. (1977). Potent uterine activity of alkaloid vasicine. Indian J. Med. Res. 66, 865-871.
22—Chandhoke, N., Gupta, O. P. and Atal, C. K. (1978). Abortifacient activity of the alkaloid vasicine through the release of prostaglandin. J. Steroid Biochem. 9, 885.
23—Gupta, O. P., Wakhloo, R. L., Sharma, M. L. and Atal, C. K. (1979). Vasicine—a potent uterine stimulant—studies on human myometrium. J. Gynaecol. India. 29, 935-938.
24—Lahiri, P. K. and Pradiian, S. N. (1964). Pharmacological investigation of vasicinol—an alkaloid from Adhatoda vasica Nees. Indian J. Exp. Biol. 2, 219-223.
25—Litchfield, J. T. and Wilcoxon, F. J. (1949). J. Pharmacol. Exp. Ther. 96, 99-113.
26—Paget, E. (1981). In LD50 and possible alternatives. Eds. Lindgren, P., Thelestam, M. and Linquist, N. G. Also see Acta Pharmacol. Toxicol. 52 (suppl. 2), 1-14.
27—Ghosh, M. N. (1984). Fundamentals of Experimental Pharmacology, Scientific Books Agency, Calcutta, pp. 153-158 & 187-190.
28—Tapper, A. R., McKinney, S. L., Nashmi, R., Schwarz, J., Deshpande, P., Labarca, C., Whiteaker, P., Marks, M. K., Collins, P. C. and Lester, H. A. (2004). Nicotine activation of α₄* receptors sufficient for reward, tolerance and sensitization. Science, 5, 1029-1032.
29—Stuhmer, W. (1992). Electrophysiological recording from xenopus oocysts. Methods Enzymol. 207, 319-339.
30—Coe, J. W., Brooks, P. R., Vetelino, M. G., Wirtz, M. C., Arnold, E. P., Huang, J., Sands, S. B. Davis, T. I., Lebel, L. A., Fox, C. B., Shrikhande, A., Heym, J. H., Schaeffer, E., Rollema, H., Lu, Y., Mansbach, R. S., Chambers, L. K., Rovetti, C. C., Schultz, D. W., Tingley, D., III; and O'Neil, B. T. (2005). Varenicline: an α₄ β₂ nicotinic redeptor partial agonist for smoking cessation. J. Med. Chem. 48, 3474-3477.
31—Barlow, R. B. and McLeod, L. J. (1969). Some studies on cytisine and its methylated derivative. Br. J. Pharmacol. 35, 161-174.
32—Scharfenberg, G., Benndorf, S. and Kempe, G. (1971). Cytisine [Tabex]^{R} als. Medikamentöse Rancherent wö hungshilfe. Dtsch Gesund-heitsw 26, 463-465.
33—Reavill, C., Walther, B., Stolerman, I. P. and Testa, B. (1990). Behavioural and pharmacokinetics studies on nicotine, cytisine and lobeline. Neuropharmacology 29, 619-624.
34—Rose, J. E., Behm, F. M. and Westman, E. C. (1994). Mecamylamine combined with nicotine skin patch facilitates smoking cessation beyond nicotine patch treatment alone. Clin. Pharmacol. Ther. 56, 86-99.
35—Warner, C. and Shoaib, M. (2005). How does bupropion work as a smoking cessation aid? Addiction Biology 10, 219-223.
36—Fattom, A., Ennifar, S., Horwith, G., Fuller, S., Pental, P., Malin, D. and Naso, R. A nicotine conjugate vaccine for the possible treatment and prevention of nicotine addiction. 225^{th} ACS National Meeting, New Orleans, La., USA. March, Abstract Book 23-27.
37—Silverstone, T. (1992). Appetite suppressants. Drugs 43-820-36.

TABLE 1
Effect of the total extract on nicotine-induced actions in mice

| Ser. # | Treatment | Actions |
|---|---|---|
| 1 | Nicotine 1.6 mg/kg (i.p) | a) Increase in locomotor activity |
| | | b) Raising of tail |
| | | c) Tremors |
| | | d) Shaking of the body |
| | | e) Increase in respiratory rate |
| | | f) No death |
| 2 | Nicotine 3.2 mg/kg (i.p) | a) Tremors |
| | | b) Fanning of tail |
| | | c) Convulsions (Clonic-type) |
| | | d) Tremors |
| | | e) Salivation |
| | | f) No death |
| 3 | Nicotine 6.4 mg/kg (i.p) | a) Circular movements |
| | | b) Convulsions |
| | | c) Death after 3 minutes |
| 4 | Total extract 1 g/kg (i.p) | No clear actions |
| | for 5 minutes | |
| 5 | Total extract 1 g/kg (i.p) + | a) Rapid respiration |
| | nicotine 1.6 mg/kg (i.p) | b) Tremors |
| | | c) Shaking of tail |
| | | d) Convulsions |
| | | e) Loss of righting reflex |
| | | f) Death after 3 minutes |

TABLE 2
Effect of total alcoholic extract on nicotine and food consumption in rats (N = 6 per group)

| | Concentration of | Quantity of | Concentration of | Liquid | Quantity of | | Quantity of | |
|---|---|---|---|---|---|---|---|---|
| | the extract in | extract consumed | nicotine in | consumed daily | nicotine | | food consumed | |
| | drinking water | daily per 200 g | drinking water | per 200 g rat | consumed | | per 200 g rat | |
| Treatment | mg/100 ml | rat mg^{a} | μg/ml | per day ml | per day mg | % decrease | per day g | % decrease |
| Control | — | — | 40 | 26.1 ± 1.3 | 1.04 | — | 23.9 ± 0.9 | — |
| Total | 600 | 19.2 | 40 | 3.2 ± 0.2* | 0.128 | 87.7 | 3.6 ± 0.12* | 84.9 |
| alcoholic | | | | | | | | |
| extract | | | | | | | | |

^{a}Calculated from the daily consumed liquid
*p < 0.01, N = 6

TABLE 3
Effect of the different fractions of the total extract on nicotine and food consumption in rats

| | Concentration of | Quantity of | Concentration of | Liquid | Quantity of | | Quantity of | |
|---|---|---|---|---|---|---|---|---|
| | the extract in | extract consumed | nicotine in | consumed daily | nicotine | | food consumed | |
| | drinking water^{a} | daily per 200 g | drinking water | per 200 g | consumed | | per 200 g rat | |
| Treatment | mg/100 ml | rat^{b} mg | μg/ml | rat ml | per day mg | % decrease | per day g | % change |
| Control | — | — | 40 | 26.7 ± 1.1 | 1.068 | — | 24.9 ± 0.9 | — |
| Fraction | 64 | 16.64 | 40 | 26 ± 0.6 | 1.04 | 2.7 | 24.3 ± 1.3 | ↓ 2.4% |
| AT-1 | | | | | | | | |
| Fraction | 5.5 | 1.38 | 40 | 25.2 ± 1.2 | 1.008 | 5.6 | 24 ± 1.2 | ↓ 3.6% |
| AT-2 | | | | | | | | |
| Fraction | 2 | 0.524 | 40 | 26.2 ± 0.4 | 1.048 | 1.87 | 25.1 ± 1.7 | — |
| AT-3 | | | | | | | | (↑ 0.8%) |
| Fraction | 48 | 12.67 | 40 | 26.4 ± 0.8 | 1.056 | 1.12 | 25.5 ± 1.4 | — |
| AT-4 | | | | | | | | (↑ 2.4%) |

^{a}Calculated and chosen in relation to the % of the fraction in the total extract.
^{b}Calculated with reference to the daily consumption.

TABLE 4
Effect of higher concentrations of the different fractions of the total Extract on nicotine and food consumption in rats

| | Concentration of | Quantity of | Concentration of | Liquid | Quantity of | | Quantity of | |
|---|---|---|---|---|---|---|---|---|
| | the extract in | extract consumed | nicotine in | consumed daily | nicotine | | food consumed | |
| | drinking water | daily per 200 g | drinking water | per 200 g | consumed | | per 200 g rat | |
| Treatment | mg/100 ml | rat^{b} mg | μg/ml | rat ml | per day mg | % decrease | per day g | % change |
| Control | — | — | 40 | 26.7 ± 0.9 | 1.068 | — | 24.9 ± 0.3 | — |
| Fraction | 330 | 84.15 | 40 | 25.5 | 1.02 | 4.5 | 23.2 ± 0.2 | ↓ 6.8% |
| AT-1 | | | | | | | | |
| Fraction | 450^{a} | 75.60 | 40 | 16.8 ± 0.2* | 0.672 | 37.1 | 18.1 ± 0.1* | ↓ 27.3% |
| AT-2 | | | | | | | | |
| Fraction | 230 | 59.56 | 40 | 25.9 ± 1.3 | 1.036 | 3 | 27.2 ± 0.6 | — |
| AT-3 | | | | | | | | (↑ 9.2%) |
| Fraction | 360 | 93.96 | 40 | 26.1 ± 1.7 | 1.044 | 2.2 | 26.9 ± 0.4 | — |
| AT-4 | | | | | | | | (↑ 8%) |

^{a}From table 3, fraction AT-2 seemed to contain the highest activity. Thus, a dose equivalent to 75% of the effective dose of the total extract was chosen and tried.
^{b}Quantity was calculated by reference to the daily drinking liquid consumption.
*p < 0.05, N = 6

TABLE 5
Effect of peganine (vasicine) on nicotine and food consumption in rats

| | Concentration of | Quantity of | Concentration of | Liquid | Quantity of | | Quantity of | |
|---|---|---|---|---|---|---|---|---|
| | the extract in | extract consumed | nicotine in | consumed daily | nicotine | | food consumed | |
| | drinking water | daily per 200 g | drinking water | per 200 g rat | consumed | | per 200 g rat | |
| Treatment | mg/100 ml | rat mg | μg/ml | per day ml | per day mg | % decrease | per day g | % decrease |
| Control | — | — | 40 | 26.25 ± 0.9 | 1.05 | — | 25.5 ± 1.4 | — |
| peganine | 140 | 7.7 | 40 | 5.5 ± 0.13* | 0.22 | 79 | 5.66 ± 0.3* | 77.8 |

*p < 0.01, N = 6

1- The use of the extract of Anisotes trisulcus (Forssk.) Vahl, family Acanthaceae in any pharmaceutical form as a method to limit tobacco consumption either via smoking, sublingually or snuffing.

2- The use of peganine in any pharmaceutical form as a means to limit tobacco consumption in any form smoking, sublingually or snuffing.

3- The use of the extract of Anisotes trisulcus (Forssk.) Vahl, family Acanthaceae in any pharmaceutical form as a means to suppress food intake.

4- The use of peganine in any pharmaceutical form as an anorxigenic agent.
